(19) United States
(12) Patent Application Publication
(10) Pub. No.: US 2017/0091378 A1
(43) Pub. Date: Mar. 30, 2017

(54) USE OF RECURRENT COPY NUMBER VARIATIONS IN THE CONSTITUTIONAL HUMAN GENOME FOR THE PREDICTION OF PREDISPOSITION TO CANCER

(71) Applicant: PHARMACOGENETICS LIMITED, Hong Kong (CN)

(72) Inventors: Hong XUE, Hong Kong (CN); Xiaofan DING, Hong Kong (CN); Shui-Ying TSANG, Hong Kong (CN)

(21) Appl. No.: 15/126,866

(22) PCT Filed: Mar. 19, 2015
(86) PCT No.: PCT/CN2015/074606

§ 371 Date: Sep. 16, 2016

Related U.S. Application Data
(60) Provisional application No. 61/968,140, filed on Mar. 20, 2014.
(60) Provisional application No. 61/990,389, filed on May 8, 2014.

(51) Int. Cl. G06F 19/18; C12Q 1/68

(52) U.S. Cl. CPC G06F 19/18; C12Q 1/6886; C12Q 2600/156

(57) ABSTRACT

In this invention, prediction on the predisposition of a human test subject to cancer is made based on machine learning-assisted comparison of the copy number variations (“CNV”) found in the constitutional DNA of the test subject with a set of diagnostic recurrent CNV features (viz. markers) selected from a collection of constitutional DNA samples from noncancer subjects (designated as “Noncancer DNA” samples) plus constitutional DNA samples from cancer patients (designated as “Cancer DNA” samples), all from the same ethnic group as the test subject. Selection and testing of the set of diagnostic recurrent CNV features is performed using a machine learning procedure, exemplified by the CFS-based method, the Frequency-based method and the Classifier-based method, together with the Naïve Bayes classification method. Prediction of the test subject's predisposition to cancer is also performed with the Naïve Bayes classification method. The cancer patients from whom the constitutional “Cancer DNA” samples are prepared, for the purpose of selection of the diagnostic recurrent CNV features, can consist of patients inflicted with one type of cancer or more than one types of cancers.

### BACKGROUND

The present invention relates to a method of using recurrent copy number variations (“CNV”) in the constitutional, viz. germline, genome of a human subject to predict the subject's predisposition to cancer. This method identifies the recurrent constitutional CNVs in a collection of DNA samples comprising both the DNA of noncancerous tissues of individuals without experience of cancer (referred to as “Noncancer DNA” samples) and the DNA of noncancerous tissues of cancer patients (referred to as “Cancer DNA” samples”), and selects from this collection using machine learning procedures a set of diagnostic recurrent CNV features comprising some of the CNVs that are enriched in individuals without experience of cancer relative to cancer patients, along with some of the CNVs that are enriched in cancer patients relative to individuals without experience of cancer, all of the same ethnic group. The usefulness of such a set of diagnostic recurrent CNV features as classifier between known “Noncancer DNA” samples and “Cancer DNA” samples is tested. Upon confirmation of usefulness, the CNVs found in the constitutional DNA of any test subject from the same ethnic group as the sources of the “Noncancer DNA” and “Cancer DNA” can be analyzed to determine the presence or absence of the various CNVs contained in the set of diagnostic recurrent CNV features, and thereby arrive at a prediction of the level of predisposition of the test subject to cancer.

The CNVs present in the DNA of the constitutional genome in noncancerous tissues of any noncancer individual, cancer patient or test subject can be determined from single nucleotide polymorphism (SNP) microarrays of human genomic DNA, qPCR, whole-genome sequencing of the person's genome, or from DNA sequencing of a subset of sequences amplified from the genome exemplified by an “AluScan” sequence subset containing inter-Alu and/or Alu-proximal genomic sequences that have been amplified by polymerase chain reaction (“PCR”) employing PCR primers the sequences of which are based on the consensus sequences of Alu-insertion elements in the human genome. The CNVs that are found in any collection of DNA samples can be identified as “recurrent” CNVs or “rare” CNVs based on their frequencies and statistical criteria. Hitherto although various “rare” CNVs have been correlated with different specific types of cancer, no correlation between recurrent constitutional CNV and cancer has been obtained and employed as a basis for the prediction of predisposition to cancer.

In the present method, the prediction of the predisposition to cancer of test subjects requires a set of diagnostic recurrent CNV features selected from the recurrent CNVs that are present in a collection of “Noncancer DNA” samples and “Cancer DNA” samples from the constitutional genomes in the noncancerous tissues of individuals without experience of cancer and cancer patients respectively. For this purpose, machine learning-assisted selection is performed using statistical selection methods exemplified by, and not limited to, the following: (I) Correlation-based Feature Selection (CSF) Method; this can be used to generate CFS-based CNV-features that are highly correlated with the recurrent CNVs in either the “Noncancer DNA” class or the “Cancer DNA” class yet uncorrelated with one another, for example using CfsSubsetEval from the Weka machine learning package together with the SestFirst method (Hall M A and Smith L A, Feature subset selection: A correlation based filter approach. International Conference on Neural Information Processing and Intelligent Information Systems. New Zealand; 1997: 8555-858; Dagliyan O et al., Optimization based tumor classification from microarray gene expression data. PLoS One 2011, 6:e14579); (II) Frequency-based Method; in this method, a CNV-feature is selected by virtue of its frequency in the “Noncancer DNA” samples being significantly different from its frequency in the “Cancer DNA” samples; and (III) Classifier-based Method; in this method, CNV-features are selected by use of a classifier, for example the ClassifierSubsetEval attribute evaluator in the Weka machine learning package together with the BestFirst method (Hall M A, et al.: The WEKA Data Mining Software: An Update. SIGKDD Explorations 2009; 11: 10-18.)

The usefulness of a diagnostic set of recurrent CNV features as a classification tool to classify DNA samples between the “Noncancer DNA” and “Cancer DNA” classes can be assessed by machine learning implementation of the Naïve Bayes classification method, and receiver operating characteristic (ROC) analysis which was originally introduced to distinguish between meaningful radar signals and noise, and has since found important application in diverse fields of clinical medicine (Zweig M H and Campbell G: Receiver-operating characteristic (ROC) plots: a fundamental evaluation tool in clinical medicine. Clinical Chemistry 1993, 39:561-577; Zhou X Statistical Methods in Diagnostic Medicine. New York, USA: Wiley & Sons; 2002).

Once a set of diagnostic recurrent CNV features selected from the recurrent CNVs found in a collection of “Noncancer DNA” and “Cancer DNA” samples from an ethnic population is found to yield an ROC-AUC (ROC-area under the curve) greater than 0.5, and therefore useful as a classification tool for classifying DNA samples between the “Noncancer DNA” and “Cancer DNA” classes, it can be employed to predict the predisposition to cancer of the constitutional DNA samples from test subjects belonging to the same ethnic population.

The principle of the prediction method referred to in [0005] consists of the assembly of a Learning Band of labeled DNA samples (viz. wherein the identities of the DNA samples are known to belong to either the “Noncancer DNA” or the “Cancer DNA” class), selection of a set of diagnostic recurrent CNV-features from all the DNA samples in the Learning Band, and confirming that the set of diagnostic recurrent CNV-features selected is useful as a classifier tool for classifying unlabeled DNA samples (viz. wherein it is not known which DNA samples belong to the “Noncancer DNA” class and which to the “Cancer DNA” class) into the “Noncancer DNA” and “Cancer DNA” classes. Once usefulness is confirmed, by for example ROC analysis, the CNVs occurring in each constituent DNA sample in the Learning Band are examined to determine the presence or absence of the different CNVs of the set of diagnostic recurrent CNV features in that constituent sample. The results obtained enable the estimation of the B-value for that constituent sample on the basis of Eqn. 1, and the relative B-values of all the labeled constituent samples in the Learning Band can be ranked on a B-value scale:

$$Pr ( cancer \vert features ) = {\sum }_{i = 1}^{n} \frac{Pr ( {feature}_{i} \vert cancer ) \times Pr ( cancer )}{Pr ( {feature}_{i} )} \\ Pr ( noncancer \vert features ) = {\sum }_{i = 1}^{n} \frac{Pr ( {feature}_{i} \vert noncancer ) \times Pr ( noncancer )}{Pr ( {feature}_{i} )} \\ \begin{matrix}B = log ( \frac{Pr ( cancer \vert features )}{Pr ( noncancer \vert features )} ) \\ = log ( \frac{( {\sum }_{i = 1}^{n} Pr ( {feature}_{i} \vert cancer ) ) \times Pr ( cancer )}{( {\sum }_{i = 1}^{n} Pr ( {feature}_{i} \vert noncancer ) ) \times Pr ( noncancer )} )\end{matrix} \qquad Eqn 1$$

in which B is the log of the ratios between Pr(cancer|feature) viz. the Bayesian posterior probability of membership in the Cancer class given the CNV data of the constituent sample, and Pr(noncancer|feature) viz. the Bayesian posterior probability of membership in the Noncancer class given the CNV data of the constituent sample; Pr(features|cancer) is the likelihood function of the CNV data given membership in the Cancer class; Pr(features|noncancer) is the likelihood function of the CNV data given membership in the Noncancer class; Pr(cancer) and Pr(Noncancer) are the prior distributions of Cancer and Noncancer samples respectively in the Learning Band. The expected classification for any test sample is ‘Cancer’ if B>0, ‘Noncancer’ if B<0, or indeterminate if B=0. Accordingly, when the different samples in the Learning Band are ranked according to their B-values, the “Noncancer DNA” samples will tend to have low rankings, whereas the “Cancer DNA” samples will tend to have high rankings, on the B-value scale.
The B-value scale constructed from all the labeled Learning Band samples provides a standard B-value scale for DNA samples for the ethnic population from which the “Noncancer DNA” samples and “Cancer DNA” samples are derived. Having this standard B-value scale, the CNVs detected in the constitutional DNA of any test subject from the same ethnic population can be analyzed to determine the presence or absence of various CNV features contained in the set of diagnostic recurrent CNV features employed to construct the B-value scale, and thereupon a B-value for the test subject on the basis of Eqn. 1. By comparing the B-value of the test subject to the B-values for various constituent “Noncancer DNA” and “Cancer DNA” samples in the Learning Band, the subject's predisposition to cancer will be revealed as high (i.e. if the subject's B-value is high on the B-value scale), intermediate (i.e. if the subject's B-value is intermediate-positioned on the B-value scale), or low (i.e. if the subject's B-value is low on the B-value scale).

### SUMMARY

The present invention relates to a method using the copy number variations (“CNV”) in the constitutional genome of a human subject to predict the subject's predisposition to cancer. This method identifies the recurrent constitutional CNVs in a collection of DNA samples comprising both the DNA of noncancerous tissues of individuals without cancer or previous experience of cancer (referred to as “Noncancer DNA” samples) and the DNA of noncancerous tissues of cancer patients (referred to as “Cancer DNA” samples”), and selects from this collection by means of machine learning procedures a set of diagnostic recurrent CNV features comprising some of the recurrent CNVs that are enriched in individuals without any experience of cancer relative to cancer patients, along with some of the CNVs that are enriched in cancer patients relative to individuals without any experience of cancer, all from the same ethnic group. The usefulness of such a set of diagnostic recurrent CNV features as classifier between “Noncancer DNA” samples and the “Cancer DNA” samples is tested. Upon confirmation of usefulness, the CNVs found in the constitutional DNA of any test subject from the same ethnic group as the sources of the “Noncancer DNA:” and “Cancer DNA” samples can be analyzed to determine the presence or absence of the various CNVs contained in the set of diagnostic recurrent CNV features, and thereby arrive at a prediction of the level of predisposition of the test subject to cancer.

The selection of a set of diagnostic recurrent CNV features comprising recurrent CNVs referred to in [0007] is performed employing machine learning methods exemplified by, but not limited to, the following methods: (I) Correlation-based Feature Selection (CSF) Method; (II) Frequency-based Method; and (III) Classifier-based Method. The usefulness of the set of diagnostic recurrent CNV features selected is tested by employing the set of features as classification tool to classify known “Noncancer DNA” and “Cancer DNA” samples into the “Noncancer DNA” and “Cancer DNA” classes using the Naïve Bayes classification method, and evaluating the accuracy of the classification achieved by means of receiver-operating characteristic (ROC) analysis.

Once a set of diagnostic recurrent CNV features is found to be useful, yielding an ROC-AUC (ROC area under the curve) value greater than 0.5, the set of features can be employed to predict the predisposition to cancer of any test subject from the same ethnic population as the sources of the “Noncancer DNA” and “Cancer DNA” samples that give rise to the set of diagnostic recurrent CNV features on the basis of Bayesian posterior probability analysis.

Because the CNV features in a set of diagnostic recurrent constitutional CNV features are typically distributed with different frequencies among the “Cancer DNA” samples from patients bearing different types of cancer, the present invention can be employed not only to identify test subjects with enhanced predisposition to cancer in general, but also subjects with enhanced predispositions to specific types of cancer.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows recurrent CNVs identified from noncancerous white blood cell DNAs, representing close approximation of constitutional (germline) DNAs, using Affymetrix SNP6.0 arrays, of (A) a Caucasian cohort of Noncancer subjects and Cancer patients; and (B) a Korean cohort of Noncancer subjects and Cancer patients. In these examples, only significantly recurrent regions with lengths greater than 1 kb and less than 10 Mb, and with a q-value<0.25 were included in the analysis. Upper panel of the figure shows q values of copy number gains (“CNV-gains”), and lower panel shows q values of copy number losses (“CNV-losses”). The q values were generated by GISTIC2.0 such that a high “−log q-value” indicated a highly non-random event. The CNV-gains (marked as A-series) and CNV-losses (marked as D-series) selected for inclusion in the CFS-based diagnostic CNV-features for the Caucasian and Korean cohorts are shown in FIG. 2 and FIG. 3 respectively.

FIG. 2 shows a set of CFS-based diagnostic recurrent CNV-features selected from the noncancerous white blood cell DNAs of a Caucasian cohort of Noncancer and Cancer subjects analyzed by Affymetrix SNP6.0 array. “Cancer Freq” indicates frequency of the CNV-feature among “Cancer DNA” samples, “Control Freq” indicates frequency of the CNV-feature among control “Noncancer DNA” samples, and “Can/Con ratio” refers to their ratio. CNVG=CNV-gain; CNVL=CNV-loss. The A-series and D-series ID numbers are added to facilitate location of the various CNV features in FIG. 1(A).

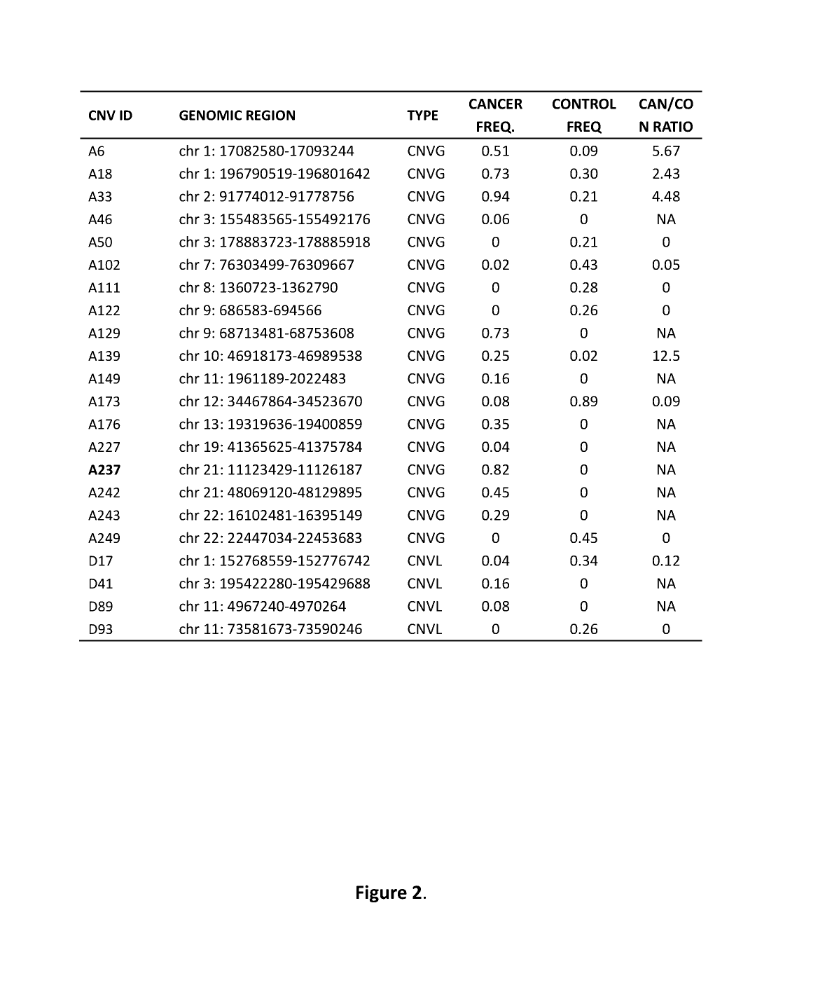

FIG. 3 shows a set of CFS-based diagnostic recurrent CNV-features selected from the noncancerous white blood cell DNAs of a Korean cohort of Noncancer and Cancer subjects analyzed by Affymetrix SNP6.0 array. “Cancer Freq” indicates frequency of the CNV-feature among “Cancer DNA” samples, “Control Freq” indicates frequency of the CNV-feature among control “Noncancer DNA” samples, and “Can/Con ratio” refers to their ratio. CNVG=CNV-gain; CNVL=CNV-loss. The A-series and D-series ID numbers are added to facilitate location of the various CNV features in FIG. 1(B).

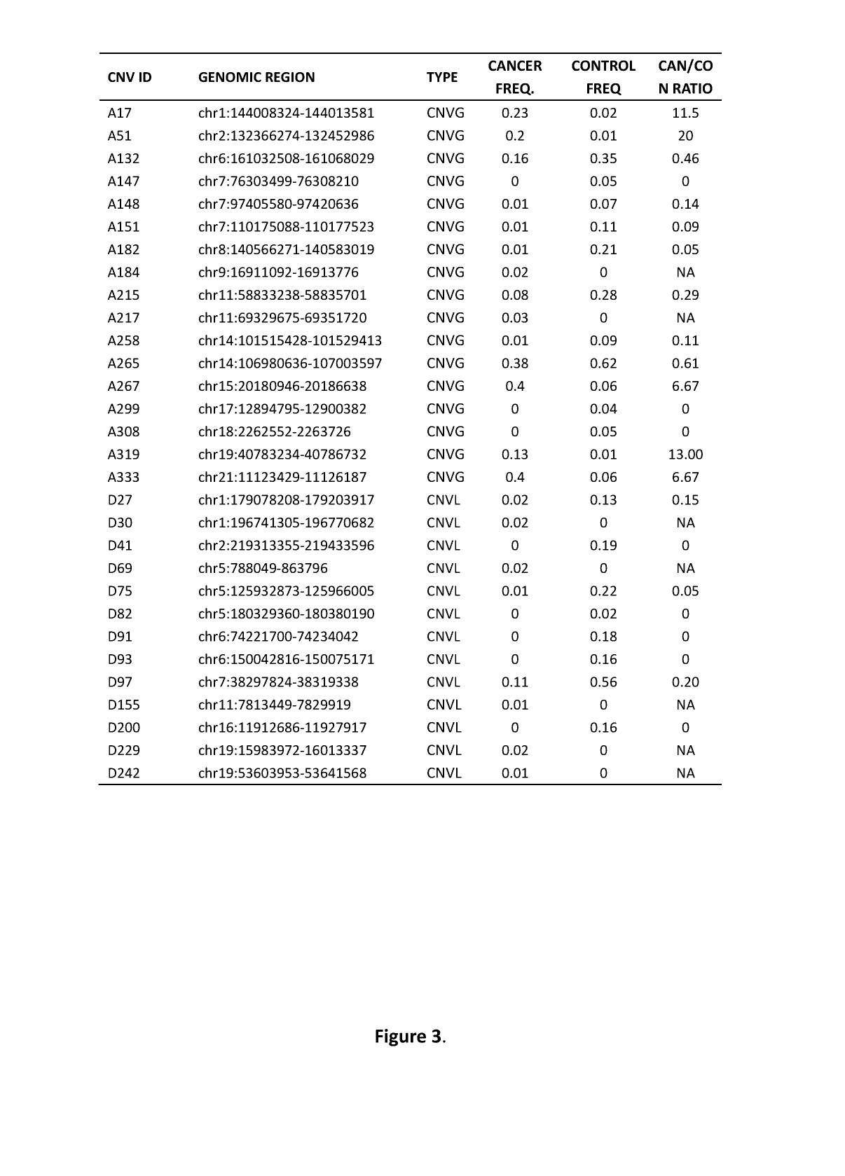

FIG. 4 shows the frequencies of occurrence of recurrent CNV-features selected by the CFS-, Frequency- and Classifier-based methods among the cancer patients and noncancer controls of (A) Caucasian cohort and (B) Korean cohort. Solid triangle, CNV-feature selected by both CFS and Frequency methods; solid circle, ones selected only by CFS method; open triangle, selected only by Frequency method; solid triangle plus solid inverted triangle, selected by CFS method, Frequency method and Classifier method; open triangle plus open inverted triangle, selected by Frequency method and Classifier method; open circle, not selected by any of the three methods. Chi-square based probability P of Cancer and Control frequencies being equal is >0.05 between the two dotted lines representing P=0.05, and <0.05 outside these two dotted lines. The two solid lines representing P′=0.05, where P′ stands for P value after Bonferroni correction, likewise separate the in-between region of P′>0.05 and the outer regions of P′<0.05.

FIG. 5 shows a table of ROC-AUC values for Caucasian and Korean samples attained with the sets of recurrent CNV-features obtained using three different CNV feature-selection methods.

FIG. 6 shows the prediction accuracies of cancer occurrence in (A) Caucasian cohort, and (B) Korean cohort, using CFS-based CNV-features. For each of the cohorts, the DNA samples were randomly separated into a Learning Band and a Test Band containing the same or approximately the same number of Noncancer DNA samples, as well as the same or approximately the same number of Cancer DNA samples. CFS-based CNV-features were selected from the Learning Band, and employed to predict the classification of each sample in the Test Band into the Noncancer and Cancer classes based on the value of B in Eqn. 1 as given in [0006]. The classification would be ‘Cancer’ if B>0, ‘Noncancer’ if B<0, or indeterminate if B=0. By repeating the random separation of samples into Learning Band and Test Band 1,000 times, and each time making predictions on every sample in the Test Band and estimating the Accuracy of prediction using Eqn. 2, 1,000 Accuracy estimates were obtained from the 1,000 runs:

$$Accuracy = \frac{\begin{matrix}[ True prediction of non \text{-} cancer ] + \\ [ True prediction of cancer ]\end{matrix}}{\begin{matrix}[ Total prediction of non \text{-} cancer ] + \\ [ Total prediction of cancer ]\end{matrix}} \times 100 \% \qquad Eqn . 2$$

The distributions of the 1,000 Accuracy estimates obtained for the Caucasian and Korean cohorts together with the Average accuracy in each case for the 1,000 runs, are indicated on graphs (A) and (B) respectively.

FIG. 7 shows the distribution of CFS-based diagnostic recurrent CNV-features in the non-tumor white blood cell DNA of (A) Caucasian cancer patients bearing different types of cancers, where the CFS-based diagnostic recurrent CNV-features are those described in FIG. 2; and (B) Korean cancer patients bearing different types of cancers, where the CFS-based diagnostic recurrent CNV-features are those described in FIG. 3. In each instance, K-means clustering was employed to cluster the different types of cancer-patient DNAs according to their contents of CFS-based CNV-features using the kmean package in R (Suzuki R, Shimodaira H. Pvclust: an R package for assessing the uncertainty in hierarchical clustering. Bioinformatics 2006, 22:1540-1542). Since the number of CFS-based CNV-features was greater than two, the CLUSPLOT function in the cluster package in R (Pilon G, et al. Displaying a clustering with CLUSPLOT. Comput Stat Data An 1999, 30:381-392) was used to reduce the dimensions of the data by principal component analysis (PCA) to produce the graphical output in terms of only the first two principal components. Different types of cancer patients included patients of colorectal cancers (green circles), gliomas (blue triangles), myelomas (red squares), gastric cancers (blue squares) and hepatocellular carcinomas (HCC, red triangles).

FIG. 8 shows a summary of the procedure in the present invention for predicting predisposition to cancer. N represents constitutional DNA samples from the noncancerous tissues of Noncancer subjects, and C represents constitutional DNA samples from the noncancerous tissues of Cancer patients.

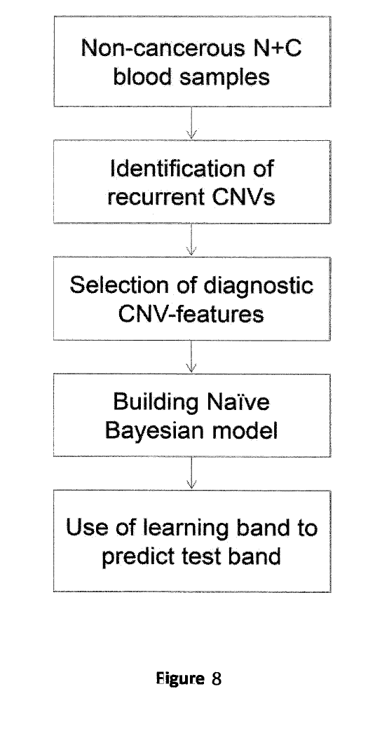

### DETAILED DESCRIPTION

It will be readily apparent to one skilled in the art that various substitutions and modifications may be made in the invention disclosed herein without departing from the scope and spirit of the invention.

### Terms

The term “a” or “an” as used herein in the specification may mean one or more. As used herein in the claim(s) the words “a” or “an” may mean one or more than one. As used herein “another” may mean at least a second or more.

The term “copy number variation”, or CNV, refers to variation from the standard human genome where the DNAs in the autosomal chromosomes, and in the X chromosome in females, are present in two copies (viz. “diploidal”), such that any DNA segment present in more than or less than two copies represents a CNV. The standard DNAs in the X and Y chromosomes in males are present in a single copy (viz. “haploidal”), such that any DNA segment present in more or less than one copy represents a CNV. Any CNV containing more than the standard number of copies constitutes a CNV-gain, and any CNV containing less than the standard number of copies constitutes a CNV-loss.

The term “recurrent CNV” refers to CNVs that are not too rare in occurrence, so that they can provide a useful basis for prediction purpose. Methods for identifying recurrent CNVs may be obtained from standard reviews such as Rueda, O. M. & Diaz-Uriarte, R. Finding Recurrent Regions of Copy Number Variation, Collection of Biostatistics Research Archive 2008, Paper 42, The Berkeley Electronic Press, which lists the MSA, GISTIC, RAE, MAR, CMAR, cghMCR, CGHregions, Master HMMs, STAC, Interval Scores, CoCoA, KC SMART, SIRAC, GEAR and Markers methods and their associated softwares.

The term “diagnostic recurrent CNV features” in the present invention refers to constitutional recurrent CNVs selected from the recurrent CNVs identified from a collection of genomic DNAs of both the noncancerous tissue samples of Noncancer (viz. noncancer individuals) subjects and the noncancerous tissue samples of Cancer (viz. cancer patients) subjects belonging to the same ethnic group. These CNV features are typically enriched in Noncancer DNAs relative to Cancer DNAs, or enriched in Cancer DNAs relative to Noncancer DNAs, such that a prediction regarding the extent of predisposition toward cancer of any test subject of the same ethnic population can be made based on the presence or absence of the various constituent diagnostic recurrent CNV features in the test subject's constitutional DNA. Selection of CNV features can be conducted using various statistical methods including but not limited to the following methods: (I) Correlation-based Feature Selection (CSF) Method, (II) Frequency-based Method, and (III) Classifier-based Method. Each of the methods gives rise to a set of diagnostic recurrent CNV features, and the utility of any set of diagnostic recurrent CNV features can be tested by employing it to classify individual samples in a sample collection comprising both labeled Noncancer DNA samples and labeled Cancer DNA samples using a probabilistic classifier such as Fisher's linear discriminant, Logistic regression, Naïve Bayes classifier, decision trees, neural networks etc. Once a set of diagnostic recurrent CNV features is found to be diagnostically useful, i.e. yielding an ROC-AUC value in excess of 0.5, it can be employed as the basis for predicting the extent of predisposition to cancer of test genomes belonging to the same ethnic population as the Noncancer and Cancer DNA samples that generated the particular set of CNV features.

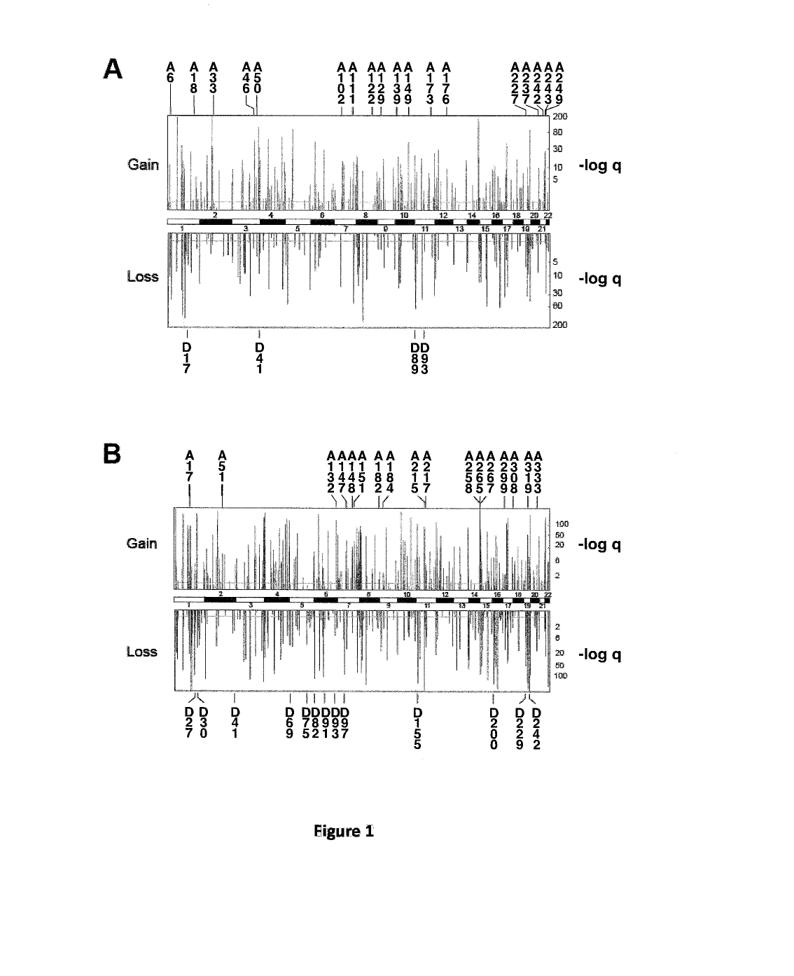

In one embodiment of the present invention, single nucleotide polymorphism (SNP) array data on whole blood samples from 51 Caucasian cancer patients and 47 ethnically-matched noncancer controls obtained using the high resolution Affymetrix SNP6.0 array platform were retrieved from the Gene Expression Omnibus (GEO) [http://www.ncbi.nlm.nih.gov/geo/] database. The program apt-copynumber-workflow with default settings from Affymetrix Power Tools (http://www.affymetrix.com/partners_programs/programs/developer/tools/powertools.affx) was employed to generate CNV callings for these Cancer and Noncancer samples using a reference template generated from the averaged microarray data for 270 HapMap samples acquired using the Affymetrix SNP6.0 platform and processed with apt-copynumber-workflow. Segmentation of neighboring copy number variations into CNV-gain segments and CNV-loss segments was performed based on the copy number values using Circular Binary Segmentation (CBS) with default parameters in DNACopy in R program (Olshen A B et al. Circular binary segmentation for the analysis of array-based DNA copy number data. Biostatistics 2004, 5:557-572). The genomic coordinates employed in the present study referred to human reference genome version hg19/GRCh37, and the annotation file used with the SNP6.0 platform was release version 32. To identify significantly recurrent CNVs, the GISTIC2.0 method (Mermel C. H. et al, Genome Biol. 12(4):R41, 2011) was employed with the options “-smallmem 1-broad 1-brlen 0.5-conf 0.9-ta 0.2-td 0.2-twosides 1-genegistic 1”. CNVs with a log 2 ratio change of either >0.2 or <−0.2 are regarded as recurrent CNVs (Ding, X. et al. Application of machine learning to development of copy number variation-based prediction of cancer risk. Genomics Insights 2014:7, 1-10). The recurrent CNVs identified are shown in FIG. 1(A).

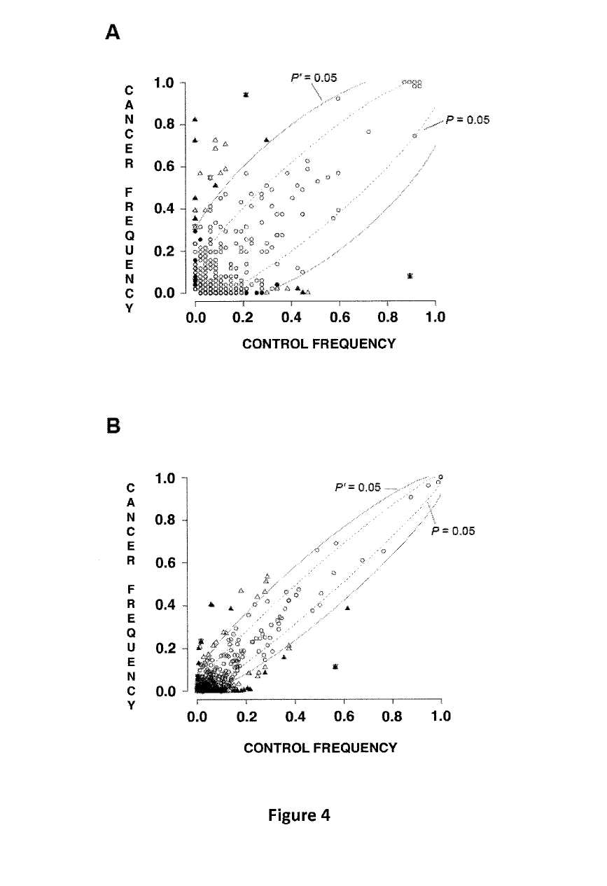

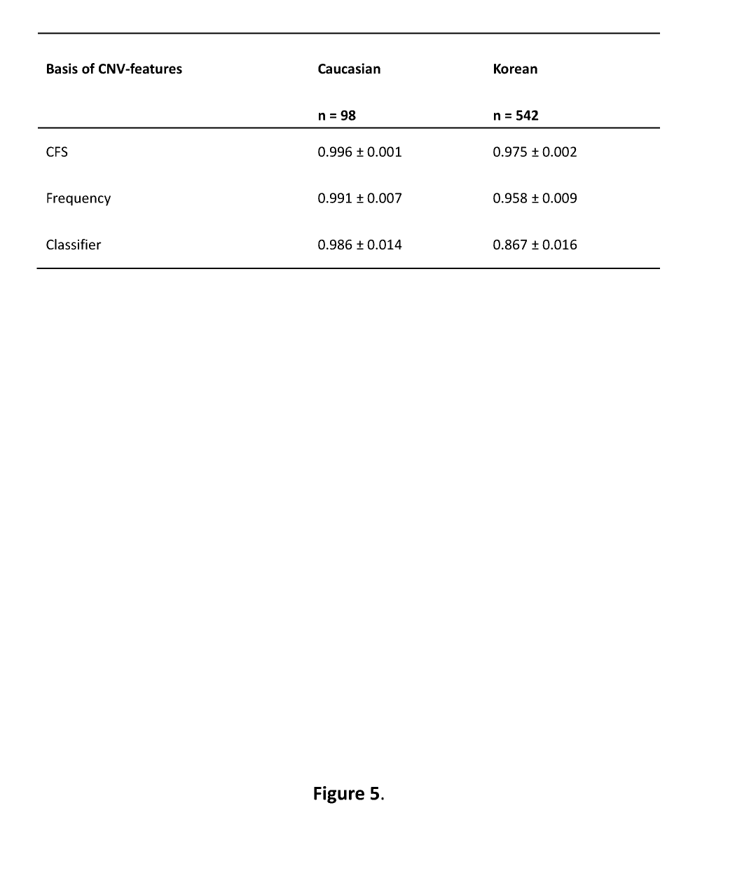

In this embodiment of the present invention, each of the Correlation-based Feature Selection (CSF) Method, Frequency-based Method, and Classifier-based Method was employed to generate three sets of diagnostic recurrent CNV features from the Caucasian Cancer and Noncancer DNA microarray data described in [0025]. To assess the capability of each of these three sets of diagnostic recurrent CNV features as a basis for classifying samples between the Cancer and Noncancer classes, the Naïve Bayes classification method from the Weka package was employed to generate a training model incorporating one of the CNV-feature sets, which was tested with 1,000 iterations of twofold cross validation. To test the robustness of the model, 10,000 permutated datasets were generated by randomly shuffling the group labels (‘Noncancer’ vs. ‘Cancer’) for each sample within the original dataset, and the whole classification process was repeated for each permutated dataset. The significance of the original classification was calculated based on the distribution of correct prediction percentage from the 10,000 permutations. The results of Naïve Bayes classification obtained using the three training models incorporating the three different CNV-feature sets to make decisions on sample classification into the ‘Noncancer’ and ‘Cancer’ classes are shown in FIG. 5, where the CNV-feature sets based on the CFS method, Frequency-based method and Classifier-based method yielded ROC-AUV values of 0.996±0.001, 0.991±0.007, and 0.986±0.014 respectively for the Caucasian samples. These high ROC-AUC values showed that all three CNV-feature ensembles are capable of classifying samples into the “Noncancer DNA” and “Cancer DNA” classes with a high level of accuracy. Each of these CNV-feature sets therefore provide a useful basis for predicting the predisposition of Caucasian test subjects to cancer. The basis for the usefulness of the sets of selected CNV-features as classifiers for the Caucasian samples is demonstrated in FIG. 4(A). The CNV features selected all displayed a highly biased distribution, occurring either frequently in the Cancer DNA samples but infrequently in the control Noncancer DNA samples, or frequently in the control Noncancer DNA samples but infrequently in the Cancer DNA samples. As a result, they are endowed with the ability to serve as markers for Cancer DNA, or as markers for control Noncancer DNA.

To confirm the expectation that CNV-feature sets can provide a valid basis for predicting predisposition to cancer, the Noncancer control DNA samples (N) in the Caucasian cohort were randomly divided in a trial run into two groupings that were equal in number when there were an even number of samples; or, when there were an odd number of samples, an extra sample was randomly allocated to one of the two groupings so that they differed in size by only a single sample. One of the groupings was randomly assigned to the Learning Band, and the other grouping to the Test Band. Similarly, for the cancer patients (C), the DNA samples from the colorectal cancer patients were randomly divided into two groupings that were either equal in size or different by only one sample; again one grouping was randomly assigned to the Learning Band, and the other to the Test Band. The glioma patient samples and the myeloma patient samples were treated the same way to finally yield an [N+C] Learning Band and an [N+C] Test Band containing an equal or near-equal number of N and C samples. Thereupon a set of CFS-based CNV-features were derived from the CNVs included in the Learning Band. Applying this set of learnt CFS-based CNV-features to each and every individual sample in the Test Band using Eqn. 1 yielded either a ‘true’ or ‘not true’ allocation of the individual into the Noncancer or Cancer class; altogether the predictions pertaining to all the individuals in the Test Band would yield an Accuracy estimate for this trial run based on Eqn. 2:

$$Accuracy = \frac{\begin{matrix}[ True prediction of non \text{-} cancer ] + \\ [ True prediction of cancer ]\end{matrix}}{\begin{matrix}[ Total prediction of non \text{-} cancer ] + \\ [ Total prediction of cancer ]\end{matrix}} \times 100 \% \qquad Eqn . 2$$

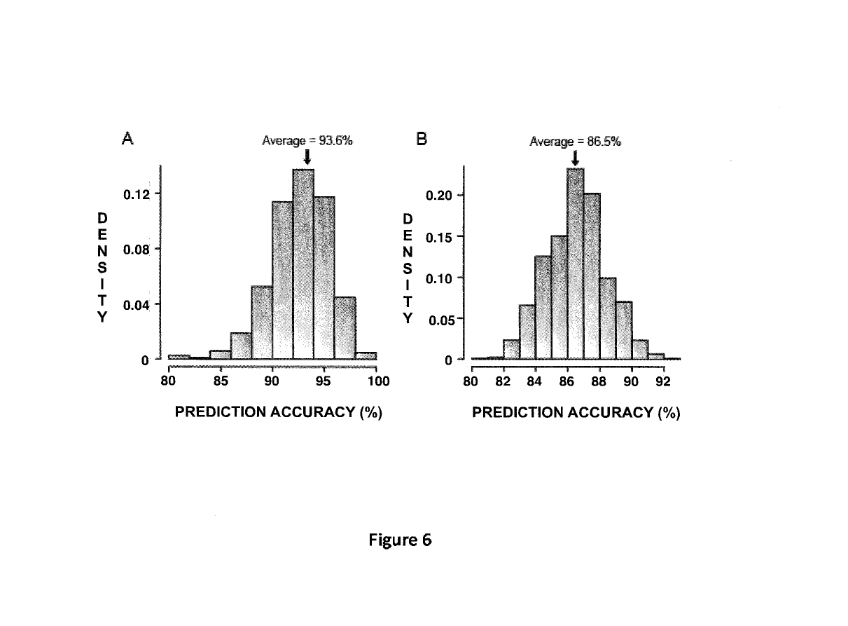

By repeating this random partition of the sample into Learning Band and Test Band 1,000 times, 1,000 estimates of accuracy were obtained. The distribution of these 1,000 accuracy estimates is shown in FIG. 6(A), and their Average value of 93.6% confirmed the valid use of diagnostic recurrent CNV features to predict the predisposition of Caucasian test subjects to cancer.

In another embodiment of the present invention, single nucleotide polymorphism array data on whole blood samples from 347 Korean cancer patients and 195 ethnically-matched Noncancer controls obtained using the high resolution Affymetrix SNP6.0 platform were retrieved from the Gene Expression Omnibus (GEO) [http://www.ncbi.nlm.nih.gov/geo/] and caArray databases [https://array.nci.nih.gov/caarray/]. Using the same procedures as those described in and [0026], recurrent CNVs comprising both CNV-gains and CNV-losses were called from the Noncancer and Cancer samples, and the Correlation-based Feature Selection (CSF) Method, Frequency-based Method, and Classifier-based Method were employed to generate three different CNV feature sets from the Noncancer and Cancer and DNA array data. The Naïve Bayes classification method was employed to generate three training model incorporating the three different CNV-feature sets, making decisions in each case on sample classification into the “Noncancer DNA” or “Cancer DNA” classes. As shown in FIG. 5, the CNV-feature sets using the CFS method, Frequency-based method and Classifier-based method yielded ROC-AUV values of 0.975±0.002, 0.958±0.009, and 0.867±0.016 respectively for the Korean samples. These high ROC-AUC values showed that all three CNV-feature ensembles are capable of classifying samples into the Noncancer and Cancer classes with a high level of accuracy, and therefore provide a useful basis for predicting the predisposition of Korean test subjects to cancer. The basis for the usefulness of the sets of selected CNV-features as classifiers for the Korean samples is demonstrated in FIG. 4(B). The CNV features selected all displayed a highly biased distribution, occurring either frequently in the Cancer DNA samples but infrequently in the control Noncancer DNA samples, or frequently in the control Noncancer DNA samples but infrequently in the Cancer DNA samples. As a result, they are endowed with the ability to serve as markers for Cancer DNA, or as markers for control Noncancer DNA.

In addition, when the various Noncancer control subjects and cancer subjects in the Korean cohort were randomly partitioned into a Learning Band and a Test Band 1,000 times as described in [0027] for the Caucasian cohort, followed by estimation of the accuracy of predictions made each time on samples in the Test Band using recurrent CNV features selected from the Learning Band by means of the CSF-based method, the distribution of the 1,000 accuracy estimates is shown in FIG. 6(B), and their Average value of 86.5% confirmed the valid use of recurrent CNV features to predict the predisposition of Korean test subjects to cancer.

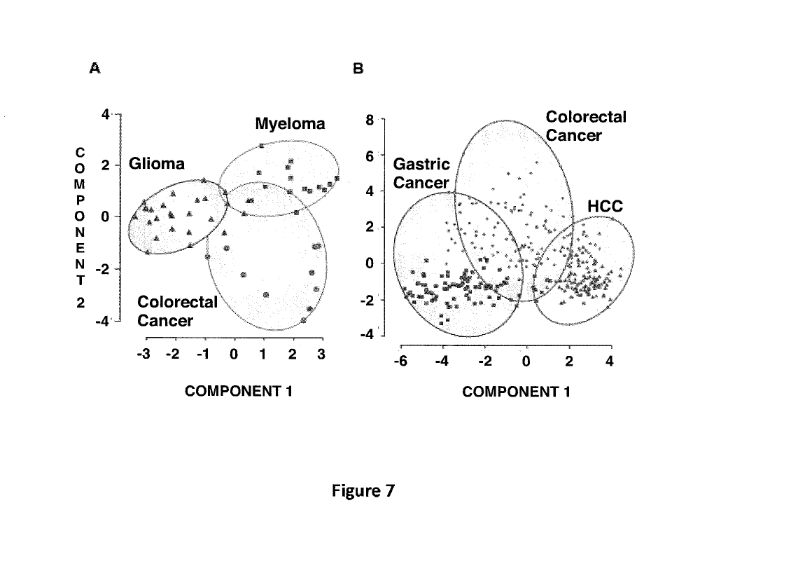

The Caucasian cancer patient samples described in [0025] came from patients inflicted variously with three types of cancers: glioma, myeloma and colorectal cancer. FIG. 7A shows that the CNV-feature contents in the three types of cancer-patient constituent genomes were dissimilar. It follows that, when carrying out the selection of diagnostic recurrent CNV features, one can employ DNAs from the noncancerous tissues of noncancer subjects, together with DNAs from the noncancerous tissues of cancer patients inflicted with one (or a restricted number of) cancer type instead of multiple cancer types, in order to focus prediction on cancer predisposition to that one (or a restricted number of) cancer type instead of predisposition to cancer in general. Likewise, the Korean cancer patient samples described in [0028] also came from patients inflicted variously with three types of cancer: gastric cancer, hepatocellular carcinoma (HCC) and colorectal cancer. As shown in FIG. 7B, the CNV-feature contents in the three types of cancer-patient constituent genomes were also dissimilar. Therefore, again one can employ DNA samples from the noncancerous tissues of noncancer subjects, together with DNA samples from the noncancerous tissues of patients inflicted with one (or a restricted number of) cancer type instead of multiple cancer types for selection of diagnostic recurrent CNV features, in order to focus prediction on cancer predisposition to that one (or a restricted number of) cancer type instead of predisposition to cancer in general. These examples indicate that the use of diagnostic recurrent CNV features to predict predisposition to cancer applies to either predisposition to cancer in general, or predisposition to one (or a restricted number of) type of cancer in particular.

1.-19. (canceled)

20. A method of using the copy number variations (“CNV”) in the constitutional (i.e. germline) genomic DNA of a human subject for predicting the predisposition of the subject to cancer, based on a comparison between the CNVs in his/her DNA with a set of diagnostic recurrent CNV features (or markers) that have been selected from the recurrent copy number variations found in a collection of constitutional DNA samples from the noncancerous tissues of noncancer subjects plus constitutional DNA samples from the noncancerous tissues of cancer patients, and comprising the steps of:
(a) Identify the recurrent copy number variations (CNV) in a collection of constitutional DNA samples from the noncancerous tissues of subjects without experience of cancer (designated as “Noncancer DNA” samples) plus constitutional DNA samples from the noncancerous tissues of cancer patients (designated as “Cancer DNA” samples), all from the same ethnic group.
(b) Select, from the recurrent CNVs in a collection of “Noncancer DNA” samples plus “Cancer DNA” samples, one or more sets of recurrent CNV features (or, markers) with the capability of serving as a classifier tool to classify DNA samples between the “Noncancer DNA” and “Cancer DNA” classes.
(c) Testing the capability of the selected set or sets of recurrent CNV features for their capability of serving as a classifier tool to classify DNA samples between the “Noncancer DNA” and “Cancer DNA” classes. Once a set of recurrent CNV features is found to be useful as a classifier tool to classify DNA samples between the “Noncancer DNA” and “Cancer DNA” classes, it can be regarded and employed as a diagnostic set of recurrent CNV features.
(d) Analyze the CNVs found in the constitutional genomic DNA in the noncancerous tissues of a test subjects belonging to the same ethnic group as the sources of the “Noncancer DNA” samples and “Cancer DNA” samples from which a diagnostic set of recurrent CNV features is derived, in order to determine the presence or absence of each and every recurrent CNV contained in the diagnostic set of recurrent CNV features. Based on the data regarding the presence or absence of the different recurrent CNVs contained in the diagnostic set of recurrent CNV features, prediction on the level of the predisposition of the test subject to cancer can then be made.

21. The method of claim 20, wherein CNVs are identified from genomic DNA based on the use of high resolution Affymetrix SNP array.

22. The method of claim 20, wherein CNVs are identified from genomic DNA based on whole genome DNA sequencing.

23. The method of claim 22, wherein the whole genome sequencing is performed with a next generation sequencing method.

24. The method of claim 20, wherein CNVs are identified from next generation sequencing of a subset of genomic DNA sequences.

25. The method of claim 24, wherein the subset of genomic DNA sequences is obtained with the use of an AluScan sequencing platform.

26. The method of claim 20, where recurrent CNVs are identified based on statistical procedures exemplified by, and not limited to, the GISTIC2.0 algorithm.

27. The method of claim 20, where recurrent CNVs are identified based on statistical procedures exemplified by, and not limited to, the AluScanCNV algorithm.

28. The method of claim 20, wherein a set of recurrent CNV features is selected from the recurrent CNVs identified in the a collection of DNA comprising both “Noncancer DNA” samples and “Cancer DNA” samples by use of a Correlation-based feature selection (CFS) method, where features are selected by virtue of their being highly correlated with either the “Noncancer DNA” class or the “Cancer DNA” class but not with one another.

29. The method of claim 20, wherein a set of recurrent CNV features is selected from the recurrent CNVs identified in the a collection of DNA comprising both “Noncancer DNA” samples and “Cancer DNA” samples by use of a Frequency-based method, where a recurrent CNV feature is selected by virtue of its frequency in “Noncancer DNA” samples being significantly different from its frequency in “Cancer DNA” samples.

30. The method of claim 20, wherein a set of recurrent CNV features is selected from the recurrent CNVs identified in the a collection of DNA comprising both “Noncancer DNA” samples and “Cancer DNA” samples by use of a Classifier-based method, where recurrent CNV features are selected by use a classifier, for example the ClassifierSubsetEval attribute evaluator from the Weka machine learning package together with the BestFirst search method.

31. The method of claim 20, wherein testing the usefulness of a set of diagnostic recurrent CNV features is performed with the use of Bayesian posterior probability analysis.

32. The method of claim 20, wherein estimation of the predisposition of a test subject to cancer is performed with the use of Bayesian posterior probability analysis.

33. The method of claim 20, wherein the “Cancer DNA” samples employed consist of the constitutional genomic DNAs of patients inflicted with more than one types of cancer.

34. The method of claim 20, wherein the “Cancer DNA” samples employed consist of the constitutional genomic DNAs of patients inflicted with a single type of cancer.

35. The method of claim 20, wherein the following recurrent CNVs are found to be individually useful as members of a set of diagnostic recurrent CNV features for predisposition to cancer testing for Caucasian test subjects (CNVG=CNV-gain; CNVL=CNV-loss):

| GENOMIC REGION | TYPE |
|---|---|
| chr 1: 17082580-17093244 | CNVG |
| chr 1: 196790519-196801642 | CNVG |
| chr 2: 91774012-91778756 | CNVG |
| chr 3: 155483565-155492176 | CNVG |
| chr 3: 178883723-178885918 | CNVG |
| chr 7: 76303499-76309667 | CNVG |
| chr 8: 1360723-1362790 | CNVG |
| chr 9: 686583-694566 | CNVG |
| chr 9: 68713481-68753608 | CNVG |
| chr 10: 46918173-46989538 | CNVG |
| chr 11: 1961189-2022483 | CNVG |
| chr 12: 34467864-34523670 | CNVG |
| chr 13: 19319636-19400859 | CNVG |
| chr 19: 41365625-41375784 | CNVG |
| chr 21: 11123429-11126187 | CNVG |
| chr 21: 48069120-48129895 | CNVG |
| chr 22: 16102481-16395149 | CNVG |
| chr 22: 22447034-22453683 | CNVG |
| chr 1: 152768559-152776742 | CNVL |
| chr 3: 195422280-195429688 | CNVL |
| chr 11: 4967240-4970264 | CNVL |
| chr 11: 73581673-73590246 | CNVL |

36. The method of claim 20, wherein the following recurrent CNVs are found to be individually useful as members of a set of diagnostic recurrent CNV features for predisposition to cancer testing for Korean test subjects (CNVG=CNV-gain; CNVL=CNV-loss):

| GENOMIC REGION | TYPE |
|---|---|
| chr1: 144008324-144013581 | CNVG |
| chr2: 132366274-132452986 | CNVG |
| chr6: 161032508-161068029 | CNVG |
| chr7: 76303499-76308210 | CNVG |
| chr7: 97405580-97420636 | CNVG |
| chr7: 110175088-110177523 | CNVG |
| chr8: 140566271-140583019 | CNVG |
| chr9: 16911092-16913776 | CNVG |
| chr11: 58833238-58835701 | CNVG |
| chr11: 69329675-69351720 | CNVG |
| chr14: 101515428-101529413 | CNVG |
| chr14: 106980636-107003597 | CNVG |
| chr15: 20180946-20186638 | CNVG |
| chr17: 12894795-12900382 | CNVG |
| chr18: 2262552-2263726 | CNVG |
| chr19: 40783234-40786732 | CNVG |
| chr21: 11123429-11126187 | CNVG |
| chr1: 179078208-179203917 | CNVL |
| chr1: 196741305-196770682 | CNVL |
| chr2: 219313355-219433596 | CNVL |
| chr5: 788049-863796 | CNVL |
| chr5: 125932873-125966005 | CNVL |
| chr5: 180329360-180380190 | CNVL |
| chr6: 74221700-74234042 | CNVL |
| chr6: 150042816-150075171 | CNVL |
| chr7: 38297824-38319338 | CNVL |
| chr11: 7813449-7829919 | CNVL |
| chr16: 11912686-11927917 | CNVL |
| chr19: 15983972-16013337 | CNVL |
| chr19: 53603953-53641568 | CNVL |
